(19) United States
(12) Patent Application Publication
(10) Pub. No.: US 2011/0015403 A1
(43) Pub. Date: Jan. 20, 2011

(54) PROCESS FOR PREPARING IODINATED AZOLES

(76) Inventor: Erasmus Vogl, Leverkusen (DE)

Correspondence Address: LANXESS CORPORATION, 111 RIDC PARK WEST DRIVE, PITTSBURGH, PA, 15275-1112, US

(21) Appl. No.: 12/223,134

(22) PCT Filed: Jan. 12, 2007
(86) PCT No.: PCT/EP2007/000236

§ 371 Date: Oct. 1, 2010

(30) Foreign Application Priority Data
Jan. 25, 2006 (DE) 10 2006 003 444.9

(51) Int. Cl. C07D 257/04; C07D 249/08

(52) U.S. Cl. 548/250; 548/269.2; 548/267.8

(57) ABSTRACT

The novel process for iodinating substituted azoles, especially for iodinating substituted 1H-tetrazoles and substituted 1H-triazoles, affords the desired compounds in high purity and with good yield.

The present invention relates to a novel process for iodinating substituted azoles, especially for iodinating substituted 1H-tetrazoles and substituted 1H-triazoles.

Azoles, especially 2- or 5-substituted 1H-triazoles and 5-substituted 1H-tetrazoles, are used, inter alia, as pharmaceutically active substances in medicine or are described, for example, as biocides for the protection of plants or industrial materials. In addition, halogenated 1H-triazoles or 1H-tetrazoles can be used as intermediates in the synthesis of related derivatives.

For the synthesis of 5-substituted 1H-triazoles and -tetrazoles, the starting materials are typically the corresponding 5-H-substituted compounds. These are typically converted to the corresponding 5-substituted derivatives by lithiation at very low temperature and treatment with an electrophile. The example which follows serves to illustrate the closest prior art.

For instance, Yoshitaka Satoh and Nicholas Marcopulos (cf. Tetrahedron Letters (1995), 36(11), 1759-62) describe a method for employing the lithiation of 1-benzyl- and 1-para-methoxybenzyltetrazoles at the 5 position. Reaction with n-butyllithium followed by treatment with electrophiles gave rise to 5-functionalized 1-benzylic tetrazoles. In summary, it can be stated that the lithiation, as the closest prior art, constitutes the method of choice for derivatization of the 5 position, for example by halogens, though the low temperature, the use of air-sensitive and relatively expensive metallation reagents such as n-butyllithium and particularly also the complete instability of the metallated intermediate even at temperatures above −78° C. are very disadvantageous.

It was an object of the present invention to provide an improved process for preparing iodinated azoles.

Surprisingly, a novel process has now been found for preparing iodinated azoles, which allows the lithiation described in the prior art and the performance of the reaction at low temperature to be avoided.

The present invention provides a process for preparing mono- or diiodinated azoles of the general formula (I) or the salts and acid addition compounds thereof

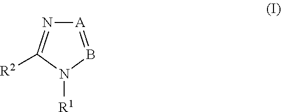

in which

### A is N, CH or CR³,

### B is N, CH or CR⁴,

with the proviso that at least one of A and B is N,
R¹ is hydrogen or in each case optionally substituted alkyl, alkenyl, alkynyl, cycloalkyl or phenyl,

### R² is I,

R³ is in each case optionally substituted alkyl, alkenyl, alicynyl, cycloalkyl, phenyl or phenethyl, and
R⁴ is in each case optionally substituted alkyl, alkenyl, alkynyl, phenyl, phenethyl or I,
by reacting compounds of the formula (II) or the salts and acid addition compounds thereof

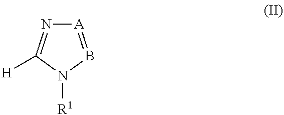

in which
A, B and R¹ are each as defined above for formula (I) with at least one oxidizing agent and elemental iodine and/or at least one iodine compound, optionally in the presence of an oxidation catalyst and optionally in the presence of a solvent or solvent mixture at a temperature between 0° C. and 200° C.

The process according to the invention preferably serves to prepare compounds of the general formula (I) in which

R¹ is hydrogen or in each case straight-chain or branched C₁—C₁₂-alkyl, C₂—C₁₂-alkenyl or C₂—C₁₂-alkynyl, or is C₃—C₈-cycloalkyl which is in each case optionally mono-to polysubstituted identically or differently by halogen; nitro; cyano; hydroxyl; Cₗ—C₆-alkoxy which is optionally mono- to nonasubstituted identically or differently by halogen; C₁—C₆-allcylthio which is optionally mono- to nona-substituted identically or differently by halogen; amino; monoalkylamino with straight-chain or branched C₁—C₆-alkyl radicals; dialkylamino with identical or different, straight-chain or branched C₁—C₆-alkyl radicals; phenyl which is optionally mono- to polysubstituted identically or differently by halogen, nitro, cyano, alkyl, haloalkyl, alkoxy, haloalkoxy, alkylthio, haloalkylthio, acyl, acyloxy, (alkoxy)carbonyl, carboxyl, amino, monoalkylamino or dialkylamino,
or
R¹ is phenyl which is optionally mono- to polysubstituted identically or differently by halogen, nitro, cyano, hydroxyl, alkyl, haloalkyl, alkoxy, haloalkoxy, alkylthio, haloalkylthio, acyl, acyloxy, alkoxycarbonyl, carboxyl, amino, monoalkylamino, dialkylamino,

### R² is I,

### A is N, CH or CR³,

where
R³ is straight-chain or branched C₁—C₈-alkyl, straight-chain or branched C₂—C₈-alkenyl, straight-chain or branched C₂—C₈-alkynyl, C₃—C₈-cycloalkyl, phenyl or phenethyl, each of which is optionally mono- to polysubstituted identically or differently by halogen, nitro, cyano, hydroxyl, alkylthio, alkoxy, amino,
and

### B is N, CH or CR⁴,

where
R⁴ is I or straight-chain or branched C₁—C₈-alkyl, straight-chain or branched C₂—C₈-alkenyl, straight-chain or branched C₂—C₈-alkynyl, C₃—C₈-cycloalkyl, phenyl or phenethyl, each of which is optionally mono- to polysubstituted identically or differently by halogen, nitro, cyano, hydroxyl, alkylthio, alkoxy, amino,
with the proviso that at least one of A and B is N.

The process according to the invention more preferably serves to prepare compounds of the general formula (1) in which

R¹ is hydrogen or straight-chain or branched C₁—C₁₂-alkyl, straight-chain or branched C₂—C₁₂-alkenyl, straight-chain or branched C₂—C₁₂-alkynyl or C₃—C₈-cycloalkyl, each of which is optionally mono- to tetrasubstituted identically or differently by fluorine; chlorine; bromine; nitro; cyano; hydroxyl; C₁—C₄-alkoxy which is optionally mono- to pentasubstituted identically or differently by fluorine, chlorine or bromine; C₁—C₄-alkylthio which is optionally mono- to pentasubstituted identically or differently by fluorine, chlorine or bromine; amino; monoalkylamino having straight-chain or branched C₁—C₄-alkyl radicals; dialkylamino with identical or different, straight-chain or branched C₁—C₄-alkyl radicals; phenyl which is optionally mono- to tetrasubstituted identically or differently by fluorine, chlorine, bromine, nitro, cyano, hydroxyl, C₁—C₄-alkyl, C₁—C₄-haloalkyl which is mono- to pentasubstituted identically or differently by fluorine, chlorine or bromine, C₁—C₄- alkoxy, C₁—C₄-halalkoxy which is mono- to pentasubstituted identically or differently by fluorine, chlorine or bromine, C₁—C₄-alkylthio, C₁—C_{1 —C}₄-haloalkylthio which is mono- to pentasubstituted identically or differently by fluorine, chlorine or bromine, C₁—C₆-acyl, C₁—C₆-acyloxy, C₁—C₆-alkoxycarbonyl, carboxyl, amino, monoalkylamino with straight-chain or branched C₁—C₄-alkyl radicals, or dialkylamino with identical or different, straight-chain or branched C₁—C₄-alkyl radicals,
or
R¹ is phenyl which is optionally mono- to tetrasubstituted identically or differently by fluorine; chlorine; bromine; nitro; cyano; hydroxyl; C₁—C₄-alkyl; C₁—C₄-haloalkyl which is mono- to pentasubstituted identically or differently by fluorine, chlorine or bromine; C₁—C₄-alkoxy, C₁—C₄-haloalkoxy which is mono- to pentasubstituted identically or differently by fluorine, chlorine or bromine;
C₁—C₄-alkylthio; C₁—C₄-haloalkylthio which is mono- to pentasubstituted identically or differently by fluorine, chlorine or bromine; C₁—C₄-acyl; C₁—C₄-acyloxy; C₁—C₄-alkoxycarbonyl; carboxyl; amino; monoalkylamino with straight-chain or branched C₁—C₄-alkyl radicals, or dialkylamino with identical or different, straight-chain or branched C₁—C₄-alkyl radicals,

### R² is I,

### A is N, CH or CR³,

where
R³ is straight-chain or branched C₁—C₈-alkyl, straight-chain or branched C₂—C₈-alkenyl, straight-chain or branched C₂—C₈-alkynyl or C₃—C₈-cycloalkyl, each of which is optionally mono- to polysubstituted identically or differently by halogen, nitro, cyano, hydroxyl, alkylthio, alkoxy, amino,
and

### B is N, CH or CR⁴,

where
R⁴ is I or straight-chain or branched C₁—C₈-alkyl, straight-chain or branched C₂—C₈-alkenyl, straight-chain or branched C₂—C₈-alkynyl or C₃—C₈-cycloalkyl, each of which is optionally mono- to polysubstituted identically or differently by halogen, nitro, cyano, hydroxyl, alkylthio, alkoxy, amino,
with the proviso that at least one of A and B is N.

The process according to the invention most preferably serves to prepare compounds of the formula (I) in which

R¹ is hydrogen, methyl, ethyl, n-propyl, isopropyl, n-butyl, isobutyl, sec-butyl, tert-butyl, n-pentyl, n-hexyl, n-heptyl, n-octyl, 2-ethylhexyl, allyl, vinyl, propargyl, where the alkyl radicals mentioned are each optionally mono- to tetrasubstituted identically or differently by fluorine, chlorine, bromine, nitro, cyano, hydroxyl, methoxy, ethoxy, n-propoxy, isopropoxy, n-butoxy, isobutoxy, sec-butoxy, tert-butoxy, trifluoromethoxy, methylthio, ethylthio, n-propylthio, isopropylthio, trifluoromethylthio, amino, methylamino, ethylamino, n-propylamino, isopropylamino, dimethylamino, diethylamino, methylethylamino, di-n-propylamino, diisopropylamino, or by phenyl which is in turn optionally mono- to trisubstituted by fluorine, chlorine, bromine, nitro, cyano, hydroxyl, methyl, ethyl, n-propyl, i-propyl, n-butyl, i-butyl, s-butyl, tert-butyl, trifluoromethyl, methoxy, ethoxy, n-propoxy, isopropoxy, n-butoxy, isobutoxy, sec-butoxy, tert-butoxy, trifluoromethoxy, methylthio, ethylthio, n-propylthio, isopropylthio, trifluoromethylthio, formyl, acetyl, acetyloxy, methoxycarbonyl, ethoxycarbonyl, carboxyl, amino, methylamino, ethylamino, n-propylamino, isopropylamino, dimethylamino, diethylamino, methylethylamino, di-n-propylamino or diisopropylamino,
or
R¹ is phenyl which is optionally mono- to trisubstituted by fluorine, chlorine, bromine, nitro, cyano, hydroxyl, methyl, ethyl, n-propyl, i-propyl, n-butyl, i-butyl, s-butyl, tert-butyl, trifluoromethyl, methoxy, ethoxy, n-propoxy, isopropoxy, n-butoxy, isobutoxy, sec-butoxy, tert-butoxy, trifluoromethoxy, methylthio, ethylthio, n-propylthio, isopropylthio, trifluoromethylthio, formyl, acetyl, acetyloxy, methoxycarbonyl, ethoxycarbonyl, carboxyl, amino, methylamino, ethylamino, n-propylamino, isopropylamino, dimethylamino, diethylamino, methylethylamino, di-n-propylamino, diisopropylamino,

### R² is I,

### A is N or CH,

and

### B is N, CH or CI,

with the proviso that at least one of A and B is N.

The process according to the invention also serves to prepare salts and acid addition compounds of the compounds of the formula (I), for example hydrohalides, hydrophosphonates or hydrosulfates, in which case the starting materials used are the corresponding salts and acid addition compounds of the formula (II).

To perform the process according to the invention, compounds of the formula (II) are used. The substituents R¹, A and B in formula (II) correspond, in their general, preferred, particularly preferred and very particularly preferred definition, to the corresponding definitions of the substituents R¹, A and B as listed above in formula (I). The compounds of the formula (II) have been known for some time to the person skilled in the art from the literature.

Suitable oxidizing agents for the performance of the process according to the invention are, for example, hydrogen peroxide, potassium peroxomonosulfate, peracids, for example peracetic acid or m-chloroperbenzoic acid, and also oxygen, excited oxygen, hypochloride, perchlorates, perborates, percarbonates, air or similar reagents containing active oxygen, or mixtures thereof.

Preference is given to using hydrogen peroxide in the form of an aqueous solution. The oxidizing agents are used generally in amounts of from 0.5 to 100 equivalents based on the azole (II). Preference is given to using from 1 to 50 equivalents based on the azole (II).

The process according to the invention is performed generally at temperatures between 0° C. and 200° C., preferably between 25° C. and 130° C. and more preferably between 60° C. and 100° C.

Useful solvents include both water alone and the common organic solvents which are not attacked by the oxidizing agents, for example petroleum ether, n-octane, n-pentane, n-hexane, cyclohexane, n-pentane, toluene, benzene, THF, diethyl ether, methyl t-butyl ether, diglyme, methanol, ethanol, isopropanol, n-butanol, tert-butanol, 2-butanol, isobutanol, n-hexanol, CH₂Cl₂, CHCl₃. It is advantageously also possible to use mixtures of two or more solvents. The solvents are preferably miscible with one another. Preference is given to using water and alcohols. Particular preference is given to using mixtures of water and methanol or ethanol or propanol or butanol or pentanol or hexanol. The different positional isomer derivatives of the alcohols are all suitable, but may afford significant differences in the yields. The water/alcohol mixing ratio may vary within wide limits; the ratio is preferably between 10:1 and 1:10.

A suitable iodine source is particularly elemental iodine. Likewise suitable are iodine depot substances such as iodine-starch compounds. Other iodine compounds such as NaI, KI, sodium periodate, iodine-oxygen acids or hypervalent iodine compounds can likewise be used. It is equally possible to use mixtures of the aforementioned iodine compounds with one another or with iodine. Iodine or the iodine compound is used in amounts between 0.1 and 10.0 equivalents, preferably between 0.4 and 2 equivalents, based on the azole.

Oxidation catalysts such as metal oxides, preferably Co, Fe or Ni complexes, for example Co-meso-tetraphenylporphin, can likewise be used to improve the yields. In this case, the catalyst is used in amounts between 0.001 and 3.0 mol % based on the azole.

The reaction can be performed at different stirrer speeds in order to ensure good mixing of the reactants.

To perform the process according to the invention, the procedure is generally to initially charge the solution or suspension of the reactant with the oxidizing agent and optionally the catalyst in a suitable solvent system and to meter in elemental iodine and/or iodine compound, in dissolved or solid form, at a suitable rate with stirring. Conversely, it is also possible first to initially charge the reactant with iodine and/or iodine compound in the solvent system and then to meter in the oxidizing agent and if appropriate the catalyst in bulk, or suitably dissolved in water or a common solvent. During or after the metered addition, the mixture is heated to a suitable temperature. Preference is given to performing the reaction within from 1 to 100 hours.

The optimal conditions depend on the substrate and its reactivity and solubility and have to be determined in each case.

For the workup, for example, the end product can be isolated by extraction and if appropriate subsequent purification steps, for example by chromatography.

The process according to the invention serves to iodinate azole compounds of the formula (I). It is also possible in an analogous manner to prepare the corresponding brominated azole compounds when, instead of elemental iodine and/or iodine compounds, elemental bromine and/or bromine compounds, for example NaBr, KBr or hydrogen bromide, are used.

The inventive method has a series of advantages over processes employed to date:

It is possible to work in very inexpensive solvents.

The reaction does not require any cooling.

Alkyl-substituted compounds can be prepared in good yields.

Iodine is required only in stoichiometric amounts, i.e. both iodine atoms of the iodine molecule are incorporated into the product.

The reagents used are available inexpensively.

The reaction can be converted easily to a large scale.

The end product is formed in a high yield and purity.

The following examples are intended to serve to illustrate the process according to the invention but without limiting it:

### EXAMPLES:

### Example 1

Synthesis of 1-(2-ethylhexyl)-5-iodo-1H-tetrazole

0.500 g of 1-(2-ethylhexyl)-1H-tetrazole (2.74 mmol) was initially charged in 10 ml of 35% H₂O₂ and 10 ml of tert-butanol, and 0.348 g of finely pulverized iodine (1.37 mmol) was added with vigorous stirring. The mixture was then stirred at 80° C. for 60 h. The reaction mixture was concentrated, taken up in a little water and extracted with ethyl acetate, and the organic phase was extracted by shaking with aqueous sodium thiosulfate. The organic phase was dried over Na₂SO₄ and then concentrated. Purification using silica gel (toluene/ethyl acetate) afforded 0.623 g of 1-(2-ethylhexyl)-5-iodo-1H-tetrazole as a colorless oil (74% yield). ¹H NMR (400 MHz, CDCl₃) δ [ppm] 0.90 (t, 3 H), 0.92 (t, 3 H), 1.30 (m, 8 H), 2.02 (m, 1 H), 4.26 (d, 2 H).

### Example 2

Synthesis of 1-(2-ethylhexyl)-5-iodo-1H-tetrazole

0.500 g of 1-(2-ethylhexyl)-1H-tetrazole (2.74 mmol) was initially charged in 10 ml of 35% H₂O₂ and 5.0 ml of tert-butanol, and 0.696 g of finely pulverized iodine (2.74 mmol) was added with vigorous stirring. The mixture was then stirred at 80° C. for 20 h. The reaction mixture was concentrated, taken up in a little water and extracted with ethyl acetate, and the organic phase was extracted by shaking with aqueous sodium thiosulfate. The organic phase was dried over Na₂SO₄ and then concentrated. Purification using silica gel (toluene/ethyl acetate) afforded 0.716 g of 1-(2-ethylhexyl)-5-iodo-1H-tetrazole as a colorless oil (85% yield). ¹H NMR (400 MHz, CDCl₃) δ [ppm] 0.90 (t, 3 H), 0.92 (t, 3 H), 1.30 (m, 8 H), 2.02 (m, 1 H), 4.26 (d, 2 H).

### Example 3

Synthesis of 1-(2-ethylhexyl)-5-iodo-1H-tetrazole

The reaction was performed as specified under example 1, except that 0.1 mol % of Co-meso-tetraphenylporphin as a catalyst, based on the tetrazole, was also metered in together with the tetrazole. The yield was 78%.

### Example 4

Synthesis of 5-iodo-1-octyl-1H-tetrazole

0.250 g of 1-octyl-1H-tetrazole (1.37 mmol) was initially charged in 5.0 ml of 35% H₂O₂ and 5.0 ml of methanol, and 0.348 g of finely pulverized iodine (1.37 mmol) was added with vigorous stirring. Thereafter, the mixture was stirred under reflux for 20 h. The reaction mixture was concentrated, taken up in a little water and extracted with ethyl acetate, and the organic phase was extracted by shaking with aqueous sodium thiosulfate. The organic phase was dried over Na₂SO₄ and then concentrated. Purification using silica gel (toluene/ethyl acetate) afforded 0.253 g of 5-iodo-1-octyl-1H-tetrazole as a pale yellow oil (60% yield). ¹H NMR (400 MHz, CDCl₃) δ [ppm] 0.90 (t, 3 H), 1.30 (m, 10 H), 1.93 (m, 2 H), 4.37 (t, 2 H).

### Example 5

Synthesis of 5-iodo-1-octyl-1H-tetrazole

0.500 g of 1-octyl-1H-tetrazole (2.74 mmol) was initially charged in 10 ml of 35% H₂O₂ and 0.696 g of finely pulverized iodine (2.74 mmol) was added with vigorous stirring. Thereafter, the mixture was stirred at 80° C. for 20 h. The reaction mixture was extracted with ethyl acetate, and the organic phase was extracted by shaking with aqueous sodium thiosulfate. The organic phase was dried over Na₂SO₄ and then concentrated. Purification using silica gel (toluene/ethyl acetate) afforded 0.389 g of 5-iodo-1-octyl-1H-tetrazole as a colorless oil (46% yield). ¹H NMR (400 MHz, CDCl₃) δ [ppm] 0.90 (t, 3 H), 1.30 (m, 10 H), 1.93 (m, 2 H), 4.37 (t, 2 H).

### Example 6

Synthesis of 1-(2-chlorobenzyl)-5-iodo-1H-1,2,4-triazole 0.250 g of 1-(2-chlorobenzyl)-1H-1,2,4-triazole (1.29 mmol) was initially charged in 5.0 ml of 35% H₂O₂ and 5.0 ml of methanol, and 0.164 g of finely pulverized iodine (0.65 mmol) was added with vigorous stirring. The mixture was then stirred at 65° C. for 20 h. The reaction mixture was concentrated, taken up in a little water and extracted with ethyl acetate, and the organic phase was extracted by shaking with aqueous sodium thiosulfate. The organic phase was dried over Na₂SO₄ and then concentrated. Purification using silica gel (toluene/ethyl acetate) afforded 0.100 g of 1-(2-chlorobenzyl)-5-iodo-1H-1,2,4-triazole as a white solid (24% yield). M.p. 98° C. ¹H NMR (400 MHz, CDCl₃) δ [ppm] 5.50 (s, 2 H), 6.86 (m, 1 H), 7.27 (m, 2 H), 7.43 (m, 1 H), 8.00 (s, 1 H).

### Example 7

Synthesis of 3-iodo-4-(3-methoxy-4-methylphenyl)-4H-1,2,4-triazole [corresponds to 5-iodo- 1-(3-methoxy-4-methylphenyl)-1H-1,3,4-triazole]

0.250 g of 4-(3-methoxy-4-methylphenyl)-4H-1,2,4-triazole (1.32 mmol) was initially charged in 5.0 ml of 35% H₂O₂ and 5.0 ml of tert-butanol, and 0.164 g of finely pulverized iodine (0.65 mmol) was added with vigorous stirring. The mixture was then stirred at 80° C. for 80 h. The reaction mixture was concentrated, taken up in a little water and extracted with ethyl acetate, and the organic phase was extracted by shaking with aqueous sodium thiosulfate. The organic phase was dried over Na₂SO₄ and then concentrated. Purification using silica gel (toluene/ethyl acetate) afforded 0.240 g of 3-iodo-4-(3-methoxy-4-methylphenyl)-4H-1,2,4-triazole as a white solid (58% yield). RF value: 0.57 (EtOAc/Et₃N =4/1). M.p.: 123° C. NMR (400 MHz, CDCl₃) δ [ppm] 2.29 (s, 3 H), 3.87 (s, 3 H), 6.73 (s, 1 H), 6.82 (d, 1 H), 7.27 (d, 1 H), 8.31 (s, 1 H).

### Example 8

Synthesis of 3,5-diiodo-4-(3-methoxy-4-methylphenyl)-4H-1,2,4-triazole [corresponds to 2,5-diiodo-1-(3-methoxy-4-methylphenyl)-1H-1,3,4-triazole]

0.250 g of 4-(3-methoxy-4-methylphenyl)-4H-1,2,4-triazole (1.32 mmol) was initially charged in 5.0 ml of 35% H₂O₂ and 5.0 ml of tert-butanol, and 0.164 g of finely pulverized iodine (0.65 mmol) was added with vigorous stirring. The mixture was then stirred at 80° C. for 80 h. The reaction mixture was concentrated, taken up in a little water and extracted with ethyl acetate, and the organic phase was extracted by shaking with aqueous sodium thiosulfate. The organic phase was dried over Na₂SO₄ and then concentrated. Purification using silica gel (toluene/ethyl acetate) afforded 0.120 g of 3,5-diiodo-4-(3-methoxy-4-methylphenyl)-4H-1,2,4-triazole as a light-colored solid (21% yield). RF value: 0.77 (EtOAc/Et₃N=4/1). M.p.: 176° C. ¹H NMR (400 MHz, CDCl₃) δ [ppm] 2.30 (s, 3 H), 3.87 (s, 3 H), 6.61 (s, 1 H), 6.74 (d, 1 H), 7.30 (d, 1 H).

What is claimed is

1. A process for iodinating substituted azoles of the general formula (I) or the salts and acid addition compounds thereof 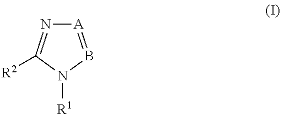
wherein
A is N, CH or CR³,
B is N, CH, CR⁴ or Cl,
wherein at least one of A and B is N,
R¹ is hydrogen or in each case optionally substituted alkyl, alkenyl, alkynyl, cycloalkyl or phenyl,
R² is I,
R³ is in each case optionally substituted alkyl, alkenyl, alkynyl, cycloalkyl, phenyl or phenethyl, and
R⁴ is in each case optionally substituted alkyl, alkenyl, alkynyl, cycloalkyl, phenyl or phenethyl,
by reacting compounds of the formula (II) or the salts and acid addition compounds thereof 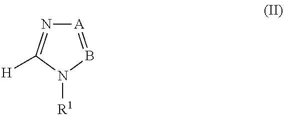
wherein
A, B and R¹ are each as defined above for formula (I) with at least one oxidizing agent and elemental iodine and/or at least one iodine compound in the presence of a solvent or solvent mixture at a temperature between 0° C. and 200° C.

2. The process according to claim 1, wherein the oxidizing agents are hydrogen peroxide, potassium peroxomonosulfate, peracids, oxygen, excited oxygen, hypochloride, perchiorates, perborates, percarbonates, air or reagents containing active oxygen, or mixtures thereof.

3. The process according to claim 1, wherein the oxidizing agent is present in an amount of from 1 to 100 equivalents based on the azole of the formula (II).

4. The process according to claim 1, wherein the elemental iodine and/or iodine compounds are present in an amount of from 0.1 to 10.0 equivalents per equivalent of azole of the formula (II).

5. The process according to claim 1, wherein the solvents used are water, alcohols, or mixtures thereof.

6. The process according to claim 1, wherein the oxidation catalyst is selected from group consisting of at least one compound Fe, Co and Ni complexes, said oxidation catalyst being present in an amount of from 0.001 to 3.0 mol % based on the azole of the formula (II).
